(19) United States
(12) Patent Application Publication
(10) Pub. No.: US 2006/0148770 A1
(43) Pub. Date: Jul. 6, 2006

(54) Combinations for the treatment of rheumatoid arithritis

(76) Inventors: Jason Fong, Philadelphia, PA (US); Edward Roydon Jost-Price, Roxbury, MA (US); Curtis Keith, Boston, MA (US); Palaniyandi Manivasakam, West Roxbury, MA (US); Grant R. Zimmermann, Somerville, MA (US); Robyn Sackeyfio, Ann Arbor, MI (US); Grant R. Zimmermann, Somerville, MA (US)

Correspondence Address: CLARK & ELBING LLP, 101 FEDERAL STREET, BOSTON, MA, 02110, US

(21) Appl. No.: 10/517,639

(22) PCT Filed: Jun. 5, 2003
(86) PCT No.: PCT/US03/17586

§ 371 Date: Jun. 28, 2005

Related U.S. Application Data
(60) Provisional application No. 60/387,528, filed on Jun. 10, 2002.

(51) Int. Cl. A61K 31/573; A61K 31/496

(52) U.S. Cl. 514/171; 514/254.07

(57) ABSTRACT

The invention features a method for treating a patient diagnosed with rheumatoid arthitis by systemically administering an azole and a steroid to the patient. The invention also features a pharmaceutical composition containing an azole and a steroid for the treatment of rheumatoid arthritis.

### BACKGROUND OF THE INVENTION

The invention relates to the treatment of rheumatoid arthritis.

One percent of humans world-wide are afflicted with rheumatoid arthritis (RA), a relentless, progressive disease causing severe swelling, pain, and eventual deformity and destruction of joints. According to the Arthritis Foundation, rheumatoid arthritis currently affects over two million Americans, of which women are three times more likely to be afflicted. Rheumatoid arthritis is characterized by inflammation of the lining of the joints and/or other internal organs, and the presence of elevated numbers of lymphocytes and high levels of proinflammatory cytokines.

Treatment of RA generally includes administration of (i) non-steroidal anti-inflammatory drugs (NSAIDs; e.g., detoprofen, diclofenac, diflunisal, etodolac, fenoprofen, flurbiprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate); (ii) steroids (e.g., cortisone, dexamethasone, hydrocortisone, methylprednisolone, prednisolone, prednisone, triamcinolone); (iii) DMARDs, i.e., disease modifying antirheumatic drugs (e.g., cyclosporine, azathioprine, methotrexate, leflunomide, cyclophosphamide, hydroxychloroquine, sulfasalazine, D-penicillamine, minocycline, and gold); or (iv) recombinant proteins (e.g., ENBREL® (etanercept, a soluble TNF receptor) and REMICADE® (infliximab) a chimeric monoclonal anti-TNF antibody).

For many years, corticosteroids have been used extensively as a first line treatment of RA. These drugs have been shown to decrease circulating monocytes and reduce macrophage phagocytosis and IL-1 secretion, resulting in inhibition of collagenase and lysosomal enzyme release (as well as reducing prostaglandin and leukotriene synthesis). Their anti-inflammatory and immunosuppressive effects provide relief for many patients and are especially useful for those patients refractory to treatment with NSAIDs. Unfortunately, corticosteroid therapy is often accompanied by numerous side effects, including bone loss, increased susceptibility to infection, osteoporosis, and peptic ulcers. Additionally, weaning patients from corticosteroids can be difficult and relapses of articular degeneration are frequent once the steroid is discontinued. Intra-articular application of these drugs has been implemented (in order to diminish the complications of oral administration) and has proven effective in reducing symptomatic joint inflammation. Nonetheless, in view of the complications associated with steroid use, it is desirable that methods employing lower doses of steroids be developed to reduce the side effects.

Because of the emerging acceptance of RA as an autoimmune disorder, much of the current therapeutic research has focused on the immune mediators associated with the development and persistence of RA. One such mediator is tumor necrosis factor-alpha (TNF-α), a cytokine produced by many cell types (especially macrophages) and known to be one of the pivotal factors initiating and maintaining the inflammatory cascade. TNF-A is thought to stimulate production amongst the cells (from RA synovial cells) of itself, IL-1, IL-6, and granulocyte-macrophage colony stimulating factor. TNF-α is also known to induce release of tissue degradative enzymes (such as matrix metalloproteinases) from both neutrophils and synoviocytes.

### SUMMARY OF THE INVENTION

We have discovered that the combination of an azole and a steroid brings about substantial suppression of TNF-α levels induced in white blood cells. TNF-α is a major mediator of inflammation. Specific blockade of TNF-α using antibodies or soluble receptors is a potent treatment for patients having rheumatoid arthritis. Therefore, suppression of TNF-α using a combination of an azole and a steroid can be used to treat rheumatoid arthritis. Moreover, based on the shared action among azole family members and among steroid family members, any member of a family can be replaced by another member of that family in the combination.

We have observed that the azole/steroid combinations of the invention result in the enhancement of the steroid activity by as much as 10-fold when it is combined with a subtherapeutic dose of an azole, even when the azole is administered at a dose lower than that known to be effective as an antifungal agent. For example, ketoconazole is often administered at 200 mg/day orally and reaches a serum concentration of about 3.2 micrograms, while prednisone is generally administered in amounts between 5-200 mg. We demonstrate that we can achieve a 10-fold increase in the potency of the steroid by combining it, at 5 mg/day, with 100 mg ketoconazole.

Accordingly, the invention features a method for treating a patient diagnosed with or at risk for developing RA in which the method consists of systemically administering to the patient an azole (e.g., an imidazole or a triazole) and a steroid (e.g., a corticosteroid, such as a glucocorticoid or a mineralocorticoid) in an amount sufficient to treat the patient. The azole and the steroid can be systemically administered within 14 days of each other (e.g., within 10 days, within five days, twenty-four hours, or one hour of each other, or even simultaneously). Administration of each compound can occur 1 to 4 times each day, or as necessary to alleviate symptoms.

The specific amounts of the azole and steroid administered depend on the specific combination of components (i.e., the specific azole/steroid combination) and can be determined by one skilled in the art.

Exemplary corticosteroids include, for example, budesonide and analogs of budesonide (e.g., budesonide (11-beta, 16-alpha(R)), budesonide (11-beta, 16-alpha(S)), flunisolide, desonide, triamcinolone acetonide, halcinonide, flurandrenolide, fluocinolone acetonide, triamcinolone hexacetonide, triamcinolone diacetate, flucinonide, triamcinolone, amcinafal, deflazacort, algestone, procinonide, flunisolide, hyrcanoside, descinolone, wortmannin, formocortal, tralonide, flumoxonide, triamcinolone acetonide 21-palmitate, and flucinolone, desonide, dexamethasone, desoximetasone, betamethasone, fluocinolide, triamcinolone, triamcinolone acetonide, triamcinolone diacetate, triamcinolone hexacetonide, beclomethasone dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, fludrocortisone, paramethasone acetate, prednisolone, prednisone, methylprednisolone, methylprednisolone acetate, methylprednisolone sodium succinate, prednisolone, prednisolone acetate, prednisolone sodium phosphate, prednisolone tebutate, clocortolone pivalate, flucinolone, dexamethasone-21-acetate, betamethasone-17-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, fluocinolone, clobetasol, cortisone, paramethasone, clocortolone, prednisolone-21-hemisuccinate free acid, prednisolone-21-acetate, prednisolone-21(-beta-D-glucuronide), prednisolone metasulphobenzoate, prednisolone terbutate, 6-alpha-methylprednisolone, 6-alpha-methylprednisolone 21-hemisuccinate sodium salt, 6-alpha-fluoroprednisolone, 6-alpha-methylprednisolone 21-acetate, 6-alpha,9-alpha-difluoroprednisolone 21-acetate 17-butyrate, prednisolone metasulphobenzoate, cortodoxone, isoprednidene, 21-deoxycortisol, prednylidene, deprodone, 6-beta-hydroxycortisol, and triamcinolone acetonide-21-palmitate. Desirably, the corticosteroid is selected from cortisone, dexamethasone, hydrocortisone, methylprenisolone, prednisone, traimcinolone, and diflorasone.

The azole can be selected from an imidazole or a triazole. Desirably, the imidazole is selected from sulconazole, miconazole, clotrimazole, oxiconazole, butocontazole, tioconazole, econazole, and ketoconazole. Desirably, the triazole is selected from itraconazole, fluconazole, voriconazole, posaconazole, ravuconazole, and terconazole.

The invention also features a method for treating a patient diagnosed with or at risk for developing rheumatoid arthritis, in which a patient is administered a first compound selected from sulconazole, miconazole, clotrimazole, oxiconazole, butocontazole, tioconazole, econazole, and ketoconazole, or itrazonazole, fluconazole, voriconazole, posaconazole, ravuconazole, and terconazole, and a second compound selected from dexamethasone, hydrocortisone, methylprednisolone, prednisone, traimcinolone, and diflorasone. In this method, the first and second compounds are administered simultaneously or within 14 days of each other, and in amounts sufficient to treat rheumatoid arthritis in the patient.

The invention also features a pharmaceutical composition that includes a pharmaceutically acceptable carrier, an azole, and a steroid, the azole and steroid being present in amounts that, when administered systemically to a patient, inhibit or reduce the symptoms of RA. Desirably, the amount of the azole present in the composition is not sufficient to act as an effective anti-fungal agent.

The invention further features a pharmaceutical composition consisting of a pharmaceutically acceptable carrier and an azole and a steroid, with the proviso that the amount of the azole present in the composition is not sufficient for the composition to be administered as an effective antifungal agent. In a preferred embodiment, the azole and steroid are present in amounts in which the activity of the steroid is enhanced at least 10-fold by the presence of the azole.

The specific amounts of the azole and steroid systemically administered to a patient or present in a pharmaceutical composition depend on the specific combination of components (i.e., the specific azole/steroid combination). Generally, when systemically administered to a human, the azole is normally administered or present in a composition at a dosage of 0.001 mg to 200 mg per day, desirably 1 mg to 100 mg per day, and most desirably 5 mg to 25 mg per day. Dosages of up to 200 mg per day may be necessary. The steroid is normally administered alone or in a composition at a dosage of about 0.1 mg to 1500 mg per day, desirably about 0.5 mg to 10 mg per day, and more desirably about 0.5 mg to 5 mg per day. Dosages of up to 3000 mg per day may be necessary.

In one embodiment of the invention, the composition contains two or more azoles and/or two or more steroid compounds. In one desired dose combination, the ratio of azole to steroid (e.g., fluconazole to glucocorticoid) is about 50:1 by weight, more desirably at least about 20:1 or 10:1 by weight, and most desirably about 4:1, 2:1, or 1:1 by weight. Low dosages of less than 10 mg and moderate dosages of between 10 mg to 20 mg of the azole, the steroid, or both can be incorporated into the pharmaceutical composition administered to the patient or used in the methods of the invention.

Compounds useful in the invention include those described herein in any of their pharmaceutically acceptable forms, including isomers such as diastereomers and enantiomers, salts, solvates, and polymorphs thereof, as well as racemic mixtures of the compounds described herein.

By “azole” is meant any member of the class of anti-fungal compounds having a five-membered ring of three carbon atoms and two nitrogen atoms (e.g., the imidazoles) or two carbon atoms and three nitrogen atoms (e.g., triazoles), which are capable of inhibiting fungal growth. A compound is considered “antifingal” if it inhibits growth of a species of fungus in vitro by at least 25%. Typically, azoles are administered in dosages of greater than 200 mg per day when used as an antifungal agent. Examples of exemplary azoles for use in the invention are described above.

By “corticosteroid” is meant any naturally occurring or synthetic steroid hormone that can be derived from cholesterol and is characterized by a hydrogenated cyclopentanoperhydrophenanthrene ring system. Naturally occurring corticosteriods are generally produced by the adrenal cortex. Synthetic corticosteriods may be halogenated. Functional groups required for activity include a double bond at Δ4, a C3 ketone, and a C20 ketone. Corticosteroids may have glucocorticoid and/or mineralocorticoid activity. Examples of exemplary corticosteroids are described above.

By “systemic administration” is meant administration of a steroid or azole by any route (e.g., oral, rectal, intravenous, intramuscular, subcutaneous, inhalation, transdermal, vaginal, intraperitoneal, interarticular or ophthahnic such that the steroid or azole is absorbed into the bloodstream of the patient.

By a “low dosage” is meant less than 10 mg per day of prednisone or equivalent, or fluconazole or equivalent. By a “moderate dosage” is meant 10 mg to 20 mg per day of prednisone or equivalent, or fluconazole or equivalent. By a “high dosage” is meant greater than about 20 mg per day of prednisone or equivalent, or fluconazole or equivalent.

By “treating” is meant administering a pharmaceutical composition for the treatment or prevention of RA. To “treat disease” or use for “therapeutic treatment” refers to administering treatment to a patient already suffering from RA to improve the patient's condition (i.e., relieve pain and inflammation, prevent joint destruction, preserve or improve functional ability, and maintain a patient's normal lifestyle). By “patient” is meant any animal (e.g., a human).

By “an effective amount” is meant the amount of a compound, in a combination of the invention, required to treat or prevent RA. The effective amount of active compound(s) used to practice the present invention for therapeutic treatment of conditions caused by or contributed to by RA varies depending upon the manner of administration, the age, body weight, and general health of the patient. Ultimately, the attending physician or veterinarian will decide the appropriate amount and dosage regimen. Such amount is referred to as an effective amount.

The combination of an azole and a steroid for the treatment of RA allows for the administration of a low dose of each compound and less total active compound, thus providing similar efficacy with less toxicity, and reduced costs. Low doses of an azole significantly increase the ability of steroids (e.g., glucocorticoids) to suppress TNF-α secretion from stimulated white blood cells and promote a significant potency shift for steroids.

Other features and advantages of the invention will be apparent from the following detailed description, and from the claims.

### DETAILED DESCRIPTION

We have discovered that the combination of an azole (e.g., an imidazole or a triazole) and a steroid (e.g., a glucocorticoid or a mineralocorticoid) has TNF-α suppressing activity that is effective for the treatment of RA. The concentration of an azole, as used in the combination, can be lower than that needed to substantially inhibit fungal growth.

Due to the TNF-α suppressing capability of the combinations, we believe that the invention is also applicable to other TNF-α mediated diseases, such as, but not limited to, stroke induced brain cell death, Sjogren's syndrome, ankylosing spondylitis, osteoarthritis, arterioscelerosis, fibromyalgia, multiple sclerosis, type 1 diabetes, systemic lupus erthrymatosis, scleroderma, and systemic sclerosis.

Antifungal azoles (e.g., imidazoles and triazoles) as described herein refer to any member of the class of anti-fungal compounds having a five-membered ring of three carbon atoms and two nitrogen atoms (imidazoles) or two carbon atoms and three nitrogen atoms (triazoles). Exemplary azoles are described above.

Corticosteroids, as described herein, refer to a class of adrenocortical hormones that include glucocorticoids, mineralocorticoids, and androgens, which are derived from cholesterol and is characterized by a hydrogenated cyclopentanoperhydrophenanthrene ring system. Exemplary corticosteroids are described above.

Therapy

Combination therapy according to the invention may be performed alone or in conjunction with another therapy and may be provided at home, the doctor's office, a clinic, a hospital's outpatient department, or a hospital. Treatment generally begins at a hospital so that the doctor can observe the therapy's effects closely and make any adjustments that are needed. The duration of the combination therapy depends on the type of disease or disorder being treated, the age and condition of the patient, the stage and type of the patient's disease, and how the patient responds to the treatment. Additionally, a person having a greater risk of developing RA (e.g., a person who is undergoing age-related hormonal changes) may receive systemic treatment to inhibit or delay the onset of symptoms.

The dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side-effects. The compounds may also be formulated together such that one administration delivers both compounds.

Formulation of Pharmaceutical Compositions

The compounds of the invention are desirably administered systemically. Suitable modes of administration include oral, rectal, intravenous, intramuscular, subcutaneous, inhalation, topical or transdermal, vaginal, intraperitoneal (IP), intraarticular, and ophthalmic.

The combination of the invention can also be provided as components of a pharmaceutical pack. The two drugs can be formulated together or separately and in individual dosage amounts. The compounds of the invention are also useful when formulated as salts.

Administration of each compound of the combination may be by any suitable means that results in a systemic concentration of the compound that, combined with the other compound, is effective for the treatment of RA. Each compound is admixed with a suitable carrier substance, and is generally present in an amount of 1-95% by weight of the total weight of the composition. The composition may be provided in a dosage form that is suitable for oral, parenteral (e.g., intravenous, intramuscular, subcutaneous), rectal, transdermal, nasal, vaginal, inhalant, or ocular administration. Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, or aerosols. The pharmaceutical compositions may be formulated according to conventional pharmaceutical practice (see, e.g., Remington: The Science and Practice of Pharmacy, (20th ed.) ed. A. R. Gennaro, 2000, Lippincott Williams & Wilkins, Philedelphia, Pa. and Encyclopedia of Pharmaceutical Technology, eds. J. Swarbrick and J. C. Boylan, 1988-2002, Marcel Dekker, New York).

Pharmaceutical compositions according to the invention may be formulated to release the active compound substantially immediately upon administration or at any predetermined time period after administration, using controlled release formulations.

Administration of compounds in controlled release formulations is useful where the compound, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of metabolism of the therapeutic compound. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Solid Dosage Forms for Oral Use

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

The two compounds may be mixed together in a tablet or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

Dosages

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

Generally, when systemically administered to a human (e.g., by oral, intramuscular, subcutaneous, topical, inhalation, rectal, vaginal and ophthalmic administration), the dosage of the azole is normally about 0.001 mg to 200 mg per day, desirably about 1 mg to 100 mg per day, and more desirably about 5 mg to 25 mg per day. Dosages up to 200 mg per day may be necessary. For intravenous administration of the azole, the dosage is normally about 1 mg to 200 mg per day, desirably about 10 mg to 150 mg per day, and more desirably about 25 mg to 50 mg per day. Systemic dosing will result in steady-state plasma concentrations of the azole of desirably 0.1 μM to 7.0 μM, more desirably, 0.5 μM to 5.0 μM, and most desirably, 1.0 μM to 2.0 μM.

The dosage range for steroids is wide, and patient response is variable. Generally, when systemically administered to a human, the dosage of the corticosteroid for use in combination with the azole is normally about 0.1 mg to 1500 mg per day, desirably about 0.5 mg to 10 mg per day, and more desirably about 0.5 mg to 5 mg per day. Dosages up to 3000 mg per day may be necessary.

The specific amounts of the azole and steroid administered depend on the specific combination of components (i.e., the specific azole/steroid combination). In a desired dose combination, the ratio of azole to steroid (e.g., fluconazole to glucocorticoid) is about 50:1 by weight, more desirably at least about 20:1 or 10:1 by weight, and most desirably about 4:1, 2:1, or 1:1 by weight.

Administration of the azole, the steroid, or both can be one to four times daily for one day to one year, and may even be for the life of the patient. Chronic, long-term administration will be indicated in many cases.

As described above, the compound in question may be systemically administered orally in the form of tablets, capsules, elixirs or syrups, or rectally in the form of suppositories, such that the azole and steroid are absorbed into the bloodstream. Parenteral administration of a compound is suitably performed, for example, in the form of saline solutions or with the compound incorporated into liposomes. In cases where the compound in itself is not sufficiently soluble to be dissolved, a solubilizer such as ethanol can be applied.

The following examples are to illustrate the invention. They are not meant to limit the invention in any way.

### Example 1

### Preparation of Pairwise Compound Mixed Combination Serial Dilution Matrix

Stock solutions of econazole, clotrimazole, diflorasone or dexamethasone (Sigma-Aldrich, St. Louis, Mo.: E4632, C6019, D8286 and D1756, respectively) were made in dimethylsulfoxide (DMSO). Using a Tom Tec Quadra Plus liquid handler, each azole was serially diluted across the columns of a 384-well master plate. Master plates were sealed and stored at −20° C. until ready for use.

The final azole and glucocorticoid combination plates were generated by transferring 1 μL from each of the azole and glucocorticoid master plates to a dilution plate containing 100 μL of media (RPMI; Gibco BRL, #11875-085), 10% Fetal Bovine Serum (Gibco BRL, #25140-097), 2% Penicillin/Streptomycin (Gibco BRL, #15140-122)) using the Tom Tec Quadra Plus liquid handler. This dilution plate was then mixed and a 10 μL aliquot transferred to the final assay plate, which had been pre-filled with 40 μL per well RPMI media containing the appropriate stimulant to activate TNF-α secretion (see below).

### Example 2

### Assay for TNF Suppressing Activity by the Combination of Azole and Steroid

The compound dilution matrix was assayed using a TNF-α ELISA method. Briefly, a 100 μL suspension of diluted human white cells contained within each well of a polystyrene 384-well plate (NalgeNunc) was stimulated to secrete TNF-α by treatment with a final concentration of 10 ng per mL phorbol 12-myristate 13-acetate (Sigma) and 750 ng per mL ionomycin (Sigma). Various concentrations of each test compound were added at the time of stimulation. After 16-18 hours of incubation at 37° C. in a humidified incubator, the plate was centrifuged and the supernatant transferred to a white opaque polystyrene 384-well plate (NalgeNunc, Maxisorb) coated with an anti-TNF antibody (PharMingen, #18631D). After a two-hour incubation, the plate was washed (Tecan PowerWasher 384) with phosphate buffered saline (PBS) containing 0.1% Tween 20 (polyoxyethylene sorbitan monolaurate) and incubated for an additional one hour with another anti-TNF antibody that was biotin labeled (PharMingen, #18642D) and horseradish peroxidase (HRP) coupled to streptavidin (PharMingen, #13047E). After the plate was washed with 0.1% Tween 20/PBS, an HRP-luminescent substrate was added to each well and light intensity measured using a LJL Analyst plate luminometer. Sets of control wells contained a serial dilution of Cyclosporin A (Sigma) starting at a final concentration of 0.5 μg per mL.

Low doses of azole significantly increased the ability of glucocorticoid to suppress TNF-α secretion from stimulated white blood cells. As seen in Table 1, econazole can greatly increase the potency of the steroid dexamethasone.

TABLE 1
Dexamethasone vs. Econazole Data
Average Result of 2 Plates
(TNF-alpha Suppression from PMA/Ionomycin-induced White Blood Cells)

| Econazole | Dexamethasone [nM] | | | | | | | | | |
|---|---|---|---|---|---|---|---|---|---|---|
| [μM] | 255.0 | 127.0 | 64.0 | 32.0 | 16.0 | 8.0 | 4.0 | 2.0 | 1.0 | 0.0 |
| 8.995 | 79.82 | 79.74 | 79.24 | 78.22 | 78.34 | 77.40 | 77.26 | 76.56 | 73.30 | 70.06 |
| 4.497 | 77.75 | 76.61 | 73.23 | 72.32 | 70.04 | 67.68 | 65.84 | 63.50 | 58.58 | 53.07 |
| 2.249 | 74.81 | 71.76 | 70.59 | 69.11 | 69.18 | 64.72 | 59.69 | 53.35 | 53.48 | 43.90 |
| 1.124 | 72.16 | 68.57 | 66.52 | 63.21 | 61.84 | 57.76 | 61.09 | 54.79 | 44.37 | 38.71 |
| 0.562 | 62.93 | 67.58 | 61.33 | 55.26 | 55.38 | 51.63 | 46.64 | 44.88 | 40.21 | 20.65 |
| 0.281 | 61.94 | 61.51 | 56.71 | 55.04 | 49.63 | 49.40 | 42.05 | 42.77 | 39.89 | 16.99 |
| 0.141 | 62.64 | 60.39 | 58.13 | 56.93 | 52.51 | 46.69 | 48.29 | 35.38 | 36.76 | 14.11 |
| 0.070 | 63.26 | 59.40 | 60.30 | 55.60 | 54.21 | 51.98 | 49.70 | 42.67 | 36.69 | 20.05 |
| 0.035 | 63.39 | 57.34 | 54.41 | 53.28 | 50.96 | 45.35 | 42.66 | 36.43 | 27.20 | 13.30 |
| 0.000 | 59.93 | 52.97 | 54.35 | 51.26 | 44.12 | 40.33 | 36.87 | 33.10 | 23.96 | 0.82 |

As a single agent, dexamethasone can suppress TNF-α secretion from phorbol 12-myristate 13-acetate and ionomycin stimulated PBMCs by 40% at a single agent concentration of 4 nM. This level of TNF-α suppression (40%) can be achieved by only 1 nM dexamethasone in the presence of 0.281 μM econazole. This represents a potency shift for the dexamethasone of 8-fold. In the presence of 2.2 μM econazole, 75% TNF-α inhibition is achieved by 255 nM dexamethasone. Furthermore, this level of activity is not achievable by dexamethasone alone (60%) even at very high concentrations that risk serious side effects. The combination of econazole and dexamethasone therefore provides a more effective and safer TNF-α suppressive therapy than steroid treatment alone.

Data from a second experiment (Table 2) confirm and extend the observed synergism between azole and glucocorticoid. Clotrimazole can greatly increase

TABLE 2
Diflorasone vs. Clotrimazole Data
Average Result of 2 Plates
(TNF-alpha Suppression from PMA/Ionomycin-induced White Blood Cells)

| Clotrimazole | Diflorasone [nM] | | | | | | | | | |
|---|---|---|---|---|---|---|---|---|---|---|
| [μM] | 120.0 | 60.0 | 30.0 | 15.0 | 7.5 | 3.8 | 1.9 | 0.9 | 0.5 | 0.0 |
| 2.000 | 64.46 | 67.62 | 63.74 | 62.41 | 58.50 | 49.03 | 44.66 | 42.20 | 37.47 | 30.02 |
| 1.000 | 59.77 | 60.27 | 63.94 | 58.23 | 56.17 | 51.82 | 43.51 | 34.93 | 36.72 | 19.90 |
| 0.500 | 62.32 | 63.70 | 61.57 | 56.74 | 53.15 | 47.13 | 43.07 | 35.79 | 39.09 | 20.07 |
| 0.250 | 60.86 | 56.42 | 57.47 | 54.78 | 48.23 | 44.03 | 35.23 | 33.85 | 28.05 | 14.29 |
| 0.125 | 53.80 | 53.05 | 51.21 | 49.59 | 45.63 | 40.93 | 31.80 | 27.24 | 25.00 | 18.40 |
| 0.063 | 53.85 | 52.03 | 51.60 | 50.37 | 41.67 | 36.07 | 30.65 | 23.39 | 23.07 | 11.43 |
| 0.031 | 48.75 | 49.50 | 51.88 | 44.79 | 39.09 | 34.94 | 26.42 | 21.88 | 14.62 | 12.47 |
| 0.014 | 51.18 | 46.54 | 45.00 | 43.83 | 34.39 | 32.26 | 20.71 | 14.99 | 9.91 | 5.86 |
| 0.008 | 51.48 | 49.38 | 44.37 | 43.62 | 40.90 | 29.99 | 25.99 | 22.17 | 15.26 | 4.92 |
| 0.000 | 47.05 | 48.95 | 49.96 | 41.37 | 36.86 | 29.01 | 21.93 | 16.12 | 9.84 | 0.35 |

the potency of the steroid diflorasone. As a single agent, diflorasone can suppress TNF-α secretion from P/I stimulated PBMCs by 29% at a single agent concentration of 3.8 nM. This level of TNF-α suppression (28%) can be achieved by only 0.5 nM diflorasone in the presence of 0.250 μM clotrimazole. This represents a potency shift for the diflorasone of 8-fold. In the presence of 2 μM clotrimazole, 65% TNF-α inhibition is achieved by 120 nM diflorasone. Furthermore, this level of activity is not achievable by diflorasone alone (47%), even at very high concentrations that risk serious side effects. The combination of clotrimazole and diflorasone would therefore provide a more effective and safer TNF-α suppressive therapy than steroid treatment alone.

### Other Embodiments

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in cellular and molecular biology, pharmacology, endocrinology, or related fields are intended to be within the scope of the invention.

What is claimed is:

1-32. (canceled)

33. A method for treating a patient diagnosed with or at risk for developing rheumatoid arthritis, said method comprising administering to the patient an azole and a steroid, wherein the azole and steroid are systemically administered simultaneously or within 14 days of each other, in amounts sufficient to treat said patient.

34. The method of claim 33, wherein said azole and steroid are administered within 10 days of each other.

35. The method of claim 34, wherein said azole and steroid are administered within five days of each other.

36. The method of claim 35, wherein said azole and steroid are administered within twenty-four hours of each other.

37. The method of claim 33, wherein said azole is an imidazole or a triazole.

38. The method of claim 33, wherein said imidazole is selected sulconazole, miconazole, clotrimazole, oxiconazole, butocontazole, tioconazole, econazole, and ketoconazole.

39. The method of claim 33, wherein said triazole is selected from itrazonazole, fluconazole, voriconazole, posaconazole, ravuconazole, and terconazole.

40. The method of claim 33, wherein said steroid is a corticosteroid.

41. The method of claim 40, wherein said corticosteroid is a glucocorticoid or a mineralocorticoid.

42. The method of claim 41, wherein said glucocorticoid is selected from cortisone, dexamethasone, hydrocortisone, methylprednisolone, prednisone, triamcinolone, and diflorasone.

43. The method of claim 33, wherein a low dosage of said azole is administered.

44. The method of claim 33, wherein a low dosage of said steroid is administered.

45. A method for treating a patient diagnosed with or at risk for developing rheumatoid arthritis, said method comprising administering to said patient:
a) a first compound selected from sulconazole, miconazole, clotrimazole, oxiconazole, butocontazole, tioconazole, econazole, and ketoconazole, or itrazonazole, fluconazole, voriconazole, posaconazole, ravuconazole, and terconazole; and
b) a second compound selected from dexamethasone, hydrocortisone, methylprednisolone, prednisone, triamcinolone, and diflorasone;
wherein said first and second compounds are administered simultaneously or within 14 days of each other, and wherein said first and second compounds are administered in amounts sufficient to treat rheumatoid arthritis in said patient.

46. A pharmaceutical composition comprising a pharmaceutically acceptable carrier and an azole and a steroid, wherein said azole and said steroid are present in an amount that, when systemically administered to a patient, inhibit or reduce the symptoms of rheumatoid arthritis; and wherein said azole is not effective as an anti-fungal agent.

47. The composition of claim 46, wherein said azole is present in an amount of 1 to 200 milligrams and said steroid is administered in an amount of 1 to 1500 milligrams.

48. The composition of claim 47, wherein said azole is present in an amount of 5 to 25 milligrams and said steroid is administered in an amount of 1 to 30 milligrams.

49. The composition of claim 46, wherein said composition comprises a low dosage of said azole.

50. The composition of claim 46, wherein said composition comprises a low dosage of said steroid.

51. A pharmaceutical composition comprising a pharmaceutically acceptable carrier and an azole and a steroid, wherein said azole is in amounts that are not effective as an antifungal agent.
